(19) United States
(12) Patent Application Publication
(10) Pub. No.: US 2019/0093068 A1
(43) Pub. Date: Mar. 28, 2019

(54) METHOD FOR MODIFYING THE MORPHOLOGY OF COAGULANT TYPE AGGREGATED FILAMENTOUS FUNGI

(71) Applicant: IFP Energies nouvelles, Rueil-Malmaison Cedex (FR)

(72) Inventors: Marion DROUGARD, Rueil Malmaison (FR); Estelle ANDRE, Rueil Malmaison (FR); Etienne JOURDIER, Bougival (FR); Mohamed Fadhel BEN CHAABANE, Paris (FR); Nicolas PANNACCI, Paris (FR)

(73) Assignee: IFP Energies nouvelles, Rueil-Malmaison Cedex (FR)

(21) Appl. No.: 16/142,924

(22) Filed: Sep. 26, 2018

(30) Foreign Application Priority Data
Sep. 27, 2017 (FR) 17/58.924

(51) Int. Cl. C12N 3/00; C12N 1/14

(52) U.S. Cl. CPC C12N 3/00; C12N 1/14

(57) ABSTRACT

The object of the invention is a method for modifying the morphology of coagulant type aggregated filamentous fungi comprising:
a) the encapsulation of the spores of the said filamentous fungi in the dispersed phase of a water-in-oil emulsion,
b) the germination of the encapsulated spores in the said dispersed phase of the emulsion,
c) the recovery of the non-aggregated germinated spores,
the said emulsion being obtained by using a microfluidic device,
   d) the culturing of the germinated spores in a liquid medium.

The present invention relates to a method for modifying the morphology of coagulant type aggregated filamentous fungi by encapsulation of spores in a microfluidic emulsion.

Filamentous fungi are currently used to produce enzymes that are capable, for example, of catalysing the conversion of biomass into ethanol in the context of producing biofuels, or producing various acids useful in the food industry. Aspergillus oryzae enables for example various enzymes (cellulases, amylases) to be produced that are useful in the biofuels production industry but also in chemistry and the food industry (example: asparaginase, which enables the level of acrylamide in foodstuffs to be reduced). Several recent publications have also shown its potential value for the production of C4 dicarboxylic acids (malate, fumarate, succinate).

The morphology of filamentous fungi is capable of having a direct impact on the productivity of the strains, principally by modifying the transport of oxygen and nutrients within the mycelium. Conventionally two types of morphology for these fungi are distinguished: a non-aggregated morphology called free mycelium, and an aggregated morphology that can appear in the form of multiple spherical aggregates, termed pellets, or in the form of a single aggregate. Depending on the species of fungi in question, the aggregation may take place at various stages of the growth, at so-called “precocious” stages before and during the termination of the spores, or at more advanced stages when the mycelium is already formed. Thus, the species in which aggregation is effective between the spores during germination are called “coagulant” type species, while those that aggregate later only during the growth of the mycelium are grouped under the “non-coagulant” type species.

Fungi occurring in aggregated forms have diffusion limitations that affect their productivity. Controlling the morphology of filamentous fungi in order to ensure the production of free mycelia would therefore represent a significant advantage in managing methods for producing enzymes that employ these fungi, so as to improve their productivity and facilitate their cultivation in a bioreactor.

At present there is no study in the literature relating to the control of the morphology of filamentous fungi with an aggregated morphology by employing microfluidic techniques.

### SUMMARY OF THE INVENTION

The present invention proposes the use of microfluidics in order to isolate physically, within an emulsion, the spores of filamentous fungi during their germination. At the end of the germination, the destabilisation of the emulsion and the recovery of the germ tubes followed by their cultivation enables a fluid growth to be obtained for certain species normally exhibiting a growth in aggregated form.

Growth in fluid form facilitates the cultivation of the fungi and enables the productivities of methods employing them to be improved.

The object of the invention is thus a method for modifying the morphology of coagulant type aggregated filamentous fungi comprising:
a) the encapsulation of the spores of the said filamentous fungi in the dispersed phase of a water-in-oil emulsion,
b) the germination of the encapsulated spores in the said dispersed phase of the emulsion,
c) the recovery of the non-aggregated germinated spores,
the said emulsion then being obtained by employing a microfluidic device,
   d) the culturing of the germinated spores in a liquid medium.

In the context of the present application, microfluidics is defined as the technology of systems that manipulate small volumes of fluids (10⁻⁹ to 10⁻¹⁸ litres), using channels the size of a few tens of micrometres.

Beneyton et al. described in 2016 the use of microfluidics for the encapsulation of spores of the species Aspergillus niger for the high-speed enzymatic screening of mutant strains by fluorescence. A large number of spores subjected to a random mutagenesis are encapsulated separately within droplets of culture medium, dispersed in a continuous oil phase. During their growth the degradation of a fluorescent substrate contained in the droplets enables the production of enzymes by the encapsulated fungus to be evaluated and thus enables the most effective mutant strains among the population to be selected. Beneyton et al. do not describe the use of this technology in order to modify the morphology of filamentous fungi.

The method according to the invention enables the morphology of filamentous fungi that naturally form aggregates to be modified by regrouping the spores during their development (=coagulant type). The object of the present invention is to prevent and even suppress this aggregation by physically isolating the spores from one another before and during the germination, by encapsulating them in a water-in-oil emulsion obtained by microfluidics.

### BRIEF DESCRIPTION OF THE DRAWINGS

FIG. 1 illustrates a typical arrangement used to form droplets at a junction in “flow-focusing”.

FIG. 2 illustrates a microfluidic device used in the examples of the invention.

FIG. 3 is a microscopic image of the encapsulation of the spores obtained by employing the method according to the invention.

FIG. 4 is a series of Erlenmeyer flask photographs of an Aspergillus oryzae culture observed under the conditions of Example 2: 4A) and 4B) illustrate a control culture, 4C) illustrates a culture of spores obtained from the encapsulation method of the invention.

FIG. 5 shows photographs obtained with a binocular magnifier of a culture of Aspergillus oryzae observed under the conditions of Example 2: 5A) sample of the control culture, 5C) sample of the culture obtained by the encapsulation method of the invention.

FIG. 6 is a series of Erlenmeyer flask photographs of a culture of Aspergillus terreus observed under the conditions of Example 3: 6A) and 6B) illustrate a control culture, 6C) illustrates a culture of spores obtained by the encapsulation method of the invention.

FIG. 7 shows photographs under a binocular magnifier of a culture of Aspergillus terreus observed under the conditions of Example 3: 7A) sample of the control culture, 7C) sample of the culture obtained by the encapsulation method of the invention.

### FILAMENTOUS FUNGI WITH AGGREGATED MORPHOLOGY

Within the context of the present application, a filamentous fungus of aggregated morphology belonging to the coagulant type is understood to mean a fungus in which the spores have not germinated and during germination naturally aggregate with one another.

According to a preferred embodiment, the filamentous fungus with aggregated morphology is selected from the species Aspergillus oryzae, Aspergillus niger or Aspergillus nidulans, and more preferably from the strains belonging to the species Aspergillus oryzae.

Spores of filamentous fungi are used in the context of the present method. The spores may be filtered before use in the method of the invention, so as to remove any mycelium that may be present.

### Encapsulation

The method for modifying the morphology of filamentous fungi according to the invention comprises a first step of encapsulation of the spores of the fungus in the dispersed phase of a water-in-oil emulsion.

The emulsion is obtained by using a microfluidic device.

The dispersed phase of the emulsion is an aqueous phase comprising at least water, spores of fungi to be encapsulated in suspension in a liquid culture medium.

The culture medium may be any medium known to the person skilled in the art to allow the germination of the filamentous fungi that are the subject matter of the present application. It may for example be a rich medium containing sources of complex nutrients (for example: yeast extract, peptone) or a minimum medium containing specified compounds (for example: mineral salts, carbonaceous and nitrogenous compounds).

It may for example be a yeast extract-peptone-dextrose medium comprising 24 g/l of potato dextrose, 5 g/l of peptone and 5 g/l of yeast extract.

The oily phase of the emulsion comprises at least one biocompatible oil that does not affect the growth of microorganisms. In particular, the oily phase comprises at least one fluorinated oil. Fluorinated oils are in fact known to be biocompatible for use with microorganisms (see for example Baret J.-C., Surfactants in droplet-based microfluidics, Lab on a chip, 2012, 12(3), p. 422).

The fluorinated oil may be chosen from perfluoroalkanes, perfluoroamines and perfluoroethers. Preferably the said fluorinated oil may be chosen from perfluoropentane, perfluorohexane, perfluorotripropylamine and perfluoropolyether.

According to a preferred embodiment the oily phase comprises a fluorinated oil, for example that marketed by the 3M company under the name Fluorinert FC-3283.

Apart from the oil the oily phase contains at least one surfactant enabling the aqueous phase droplets dispersed in the oily phase to be stabilised. The surfactant may preferably be selected from biocompatible surfactants. Preferably, the surfactant is a fluorinated surfactant, such as that marketed by the company Dolomite under the name Picosurf.

The emulsion according to the invention is obtained by microfluidics, that is to say by using a miniaturised system enabling microdroplets to be generated under flow conditions in a reproducible and controlled manner within microchannels arranged according to a particular geometric pattern.

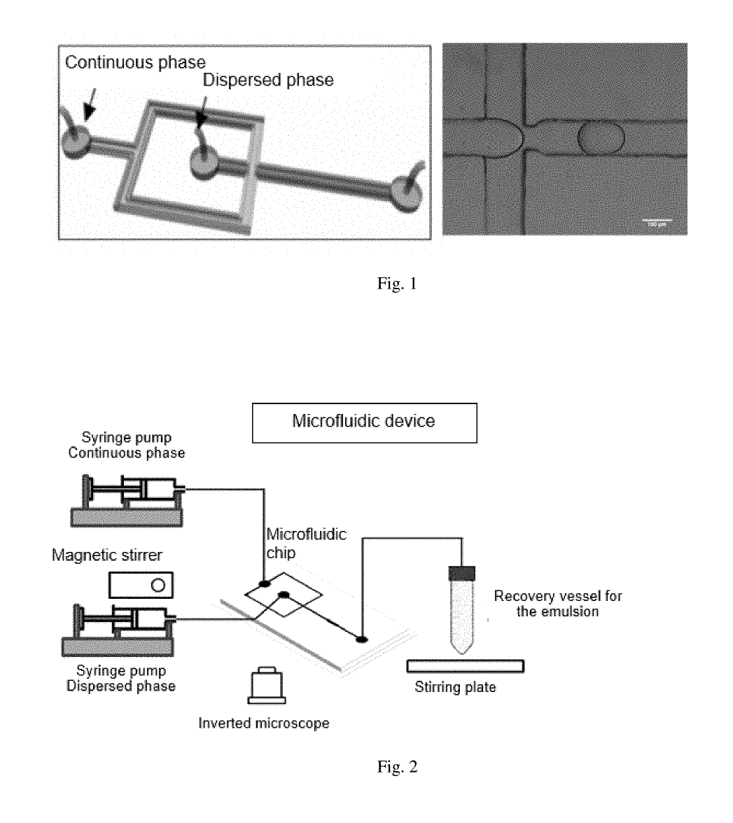

These geometric arrangements are well known to the person skilled in the art. Among the most conventional methods for generating a flow of microdroplets, there may be mentioned the T-junction and the flow-focussing geometry. According to a preferred embodiment, the emulsion according to the invention is obtained by means of a microfluidic system having a flow-focussing geometry. In this type of system the dispersed phase is injected co-linearly to two oil flows surrounding it. Depending on the selected experimental conditions, a jet of dispersed phase can form between the two oil flows. The laminar co-flow that is thereby formed passes through a constriction, which intensifies the flow and possibly causes the jet of dispersed phase to break up into droplets. In the flow-focussing geometry the droplets are thus preferably formed at a cross-shaped junction or the like. This type of system is illustrated for example in FIG. 1.

The microfluidic device according to the invention comprises in particular:
a first microfluidic conduit, fed with aqueous phase containing in suspension the spores to be encapsulated;
a second microfluidic conduit fed with oily phase;
   the first conduit leading into the second conduit and forming a fluidic junction with the latter.

Since the two liquid phases are not miscible, an interface is formed at the level of the fluidic junction, corresponding to the intersection of the microfluidic conduits, thus allowing the formation of aqueous phase droplets dispersed in the oily phase, that is to say the formation of the emulsion. The pressures and flow rates of the two liquid phases are dimensioned such that the interface remains stable due to equilibrium between the said pressures and the surface tension.

The microfluidic device according to the invention preferably also comprises at least one microfluidic conduit for removing the emulsion that is formed.

According to a preferred embodiment, the microfluidic device has a planar geometry, in particular the device according to the invention is a microfluidic chip.

The first and second microfluidic conduits of the microfluidic system used, in particular the conduits of the microfluidic chip that is used, preferably have a cross-section that can be inscribed in a circle of diameter between 1 μm and 1 mm, preferably between 50 and 500 μm, and in particular of the order of 125 μm.

The aqueous phase of the emulsion obtained by means of the device according to the invention is in the form of droplets dispersed in the oily phase, the said droplets having a diameter of between 10 μm and 1 mm, preferably between 50 and 500 μm, and more preferably between about 140 and 150 μm. The diameter of the droplets may for example be measured by any method known to the person skilled in the art, and in particular by image analysis on an optical microscope.

Such a device or a microfluidic chip can be manufactured from a substrate made of silicon, silica or glass by conventional techniques of photolithography and etching borrowed from microelectronics. As a variant, the device may be made of plastic material (in particular polydimethylsiloxane—PDMS) by moulding, from a mould, produced in turn by photolithography.

The microfluidic device according to the invention may advantageously be hydrophobically treated, for example by treatment with OTS (octadecyltrichlorosilane).

The microfluidic encapsulation device may furthermore comprise:
an injection means for injecting into the first conduit of the said device, a suspension formed by the aqueous phase and by the spores to be encapsulated; the means may be for example a syringe with a syringe pump or a pressure controller; and
an injection means, in the first conduit of said device, for injecting the oily phase; it may also be a syringe with a syringe pump or a pressure controller.

In the context of the present invention, the concentration of spores in the aqueous phase and the injection rates of the phases are adapted so as to have on average between 1 and 10 spores per droplet, preferably 1 spore per droplet.

In particular, the aqueous phase comprising the spores can be introduced into the microfluidic device at a flow rate of between 0.1 and 20 ml/h, preferably between 1 and 10 ml/h, and more preferably of the order of 3 ml/h.

The oily phase containing the surfactant may be introduced into the microfluidic system at a flow rate of between 1 and 50 ml/h, preferably between 5 and 20 ml/h, and more preferably of the order of 8 ml/h.

The encapsulation step of the method according to the invention may last at least 30 minutes, preferably from 30 minutes to 10 hours, still preferably from 1 to 5 hours, and more preferably about 3 hours.

The emulsion formed is recovered at the outlet of the microfluidic device via the microfluidic outlet conduit. The microfluidic outlet conduit has a cross-section that can be inscribed in a circle of diameter between 125 and 1500 μm, preferably between 500 and 1000 μm, and in particular of the order of 750 μm.

Unlike microfluidic systems commonly developed for example for enzymatic screening, it is not intended to recover separately each of the isolated spores in order to characterise them, or to sort them, since the population used is genetically identical.

Thus, the emulsion at the outlet of the microfluidic device is recovered in its entirety in a single container and is kept stirred to prevent it from breaking up. The stirring may for example be maintained between 50 and 300 rpm, preferably between 120 and 150 rpm, and more preferably of the order of 120 rpm, at between 20 and 30° C.

### Germination

Once the encapsulation has been performed, the method according to the invention implements a germination stage b) of the spores.

The emulsion recovered at the end of step a) is kept stirred for the time required for the germination of the spores, preferably between 1 and 40 h, preferably between 10 and 30 hours, and more preferably still about 19 hours.

The germination is carried out at ambient temperature, in particular between 20° and 30° C., preferably at 24° C.

As previously, the stirring may for example be maintained between 50 and 300 rpm, preferably between 120 and 150 rpm, and more preferably on the order of 120 rpm, at between 20 and 30° C.

### Recovery of the Germinated Spores

Once the germination has finished, the emulsion is destabilised so as to recover an aqueous phase containing the germinated spores (called germ tubes), corresponding to the start of formation of the mycelium.

The end of germination is known to the person skilled in the art. Data are available in the literature for each fungus and a microscopic observation allows it to be confirmed.

Any means known to the person skilled in the art for breaking the emulsion can be implemented.

According to a preferred embodiment, the recovery of non-aggregated germinated spores is carried out by destabilisation of the emulsion.

For example, the emulsion can be rinsed by means of an oily phase free from surfactant, until the surfactant concentration is too low to ensure the stability of the dispersion of the droplets of aqueous phase in the oil. The two phases can then easily be separated.

The aqueous phase thus recovered can be used to seed a culture on a larger scale, such as a vial, a propagation fermenter, or even a production fermenter.

### Culturing of the Germinated Spores

The method according to the invention comprises a final step d) of culturing the germinated spores in a liquid medium.

The culturing of step d) is preferably carried out with stirring between 20° and 30° C., preferably 24° C., in a medium enabling the growth of filamentous microorganisms, preferably a rich medium.

The culturing of step d) preferably lasts between 1 and 48 hours, and in particular approximately 24 hours.

The method according to the invention enables, generally after 24 hours' growth, a fluid, non-aggregated culture of mycelium of filamentous fungi to be obtained that is advantageous for their industrial implementation (facilitated propagation, better growth by facilitated diffusion of nutrients within the mycelium).

The culturing can be used as a preliminary step to the seeding of a bioreactor or any other facility allowing the propagation and growth of the encapsulated filamentous fungus according to the invention.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding French application No. 17/58.924, are incorporated by reference herein.

### EXAMPLES

The following examples illustrate the invention.

### Example 1: Protocol

### Aqueous Phase:

An aqueous phase was prepared made of 15 ml of a solution of YPDP-rich medium (potato dextrose 24 g/L, yeast extract 5 g/L and peptone 5 g/L) and was diluted to half and seeded with fungal spores at a concentration of 5×10⁵ spores/mL, in a glass syringe.

Magnetic stirring is implemented in the syringe to prevent sedimentation of the spores. The spore bank was previously filtered in order to avoid the possible presence of mycelium.

### Oily Phase:

An oily phase was prepared made of 30 mL of FC3283 fluorinated oil solution to which was added Picosurf biocompatible surfactant in an amount of 3 g/L, in a glass syringe.

### Experimental Device:

The experimental device consists of a microfluidic chip illustrated in FIG. 2, comprising microfluidic channels, obtained by crosslinking and bonding PDMS (RTV 615, Momentive company) on a glass slide by Plasma air (formation of active sites under the action of a radio frequency electric field, which produces a strong adhesion between the two surfaces. This method consists in activating the surface with plasma oxygen (or air alone). To do this, a primary vacuum is created and an excited gas is generated under the action of a radio frequency electric field. The electrons and the high-energy ions that constitute the plasma make it possible to break the surface bonds and create “active” sites, two thus treated PDMS-PDMS or PDMS-glass surfaces can then form bonds and adhere strongly).

The microfluidic chip shown in FIG. 2 is pierced at the inlet of the aqueous and oily phases, and at the outlet. The microfluidic inlet conduits have an internal diameter of 125 μm and those of the outlet an internal diameter of 750 μm.

Treatment with OTS (Octadecyltrichlorosilane) Renders the System Hydrophobic.

The microfluidic chip is placed under an inverted microscope equipped with a camera enabling the encapsulation to be viewed in real time. The two syringes containing the aqueous and oily phases are attached to two syringe pumps, allowing the separate adjustment of the flow rates of each phase. The emulsion at the outlet of the microfluidic chip is collected in a 50 mL Falcon tube kept constantly stirred at 120 rpm throughout the duration of the germination.

### Encapsulation:

The aqueous phase containing the spores is injected at a flow rate of 3 ml/h, the oily phase containing the surfactant at a flow rate of 8 ml/h. The encapsulation lasts 3 hours and enables approximately 10 ml of emulsion and 20 ml of fluorinated oil to be obtained.

### Germination:

The emulsion is maintained, while stirring at 120 rpm, at 24° C. for 19 hours, the time necessary for the spores to germinate.

### Destabilisation of the Emulsion:

At the end of the germination step, the emulsion is destabilised so as to recover only the aqueous phase containing the germinated spores. To do this, successive rinses with FC3283 oil that is free of surfactant are performed:

The residual oil at the bottom of the tube is removed, then the virgin oil is added in an amount equivalent to that of the emulsion (about 10 mL). The tube is stirred manually for 5 minutes. This operation is repeated as many times as necessary until a clear, oil-free aqueous phase containing the germinated spores is observed.

### Growth:

The aqueous phase thus recovered (approximately 8 ml) is then transferred to a 250 ml Erlenmeyer flask in the YPDP rich medium in a sufficient quantity for 50 ml.

The growth at 24° C. while stirring at 175 rpm lasts 24 hours.

### Example 2: Application to Aspergillus oryzae

The spores of Aspergillus oryzae, a species which has an aggregated type morphology in the form of pellets formed by a coagulating mechanism during its culture in a liquid rich medium, are encapsulated according to the protocol described in Example 1.

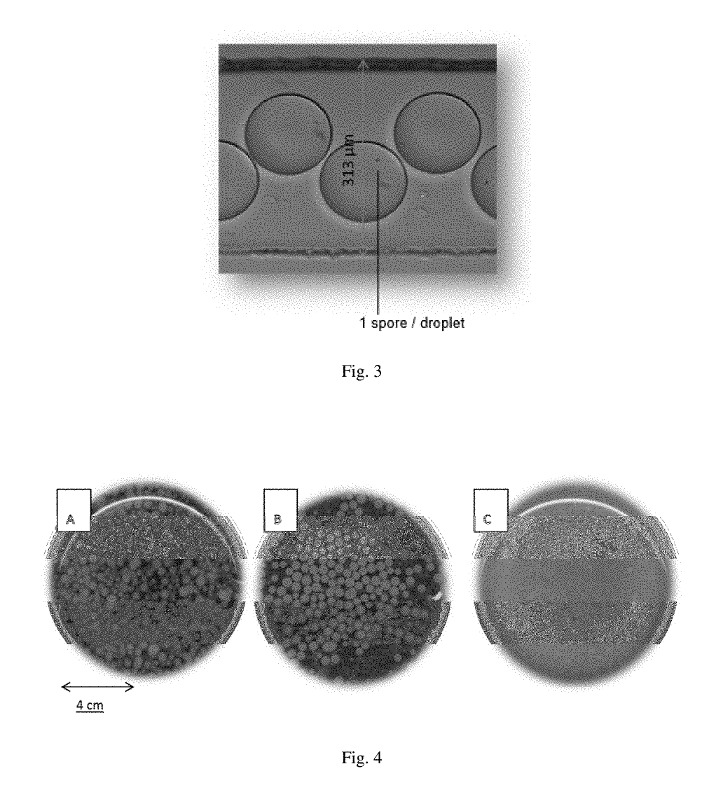

Observation of the encapsulation step with the microscope (FIG. 3) shows the presence of an average of 1 spore/droplet and a droplet size of approximately 140 to 150 μm in diameter (2 nL).

Two controls whose spores are not encapsulated according to the method of the invention are also prepared:
a control A containing 10 ml of YPDP medium diluted to half, seeded with 5×10⁵ spores/ml and placed in a Falcon tube under the same conditions as the emulsion recovery tube; and
a control B, identical to the control A, to which was added 20 ml of oil+surfactant.

After carrying out the method described in Example 1, the 10 ml of destabilised emulsion according to the invention containing the germinated spores are transferred to an Erlenmeyer flask containing 40 ml of YPDP rich medium. In the same way, the two controls are also transferred to two Erlenmeyer flasks containing 40 ml of YPDP rich medium.

After 24 hours' growth, the strain of Aspergillus oryzae tested (NRRL 3488) shows the morphologies presented in FIG. 4.

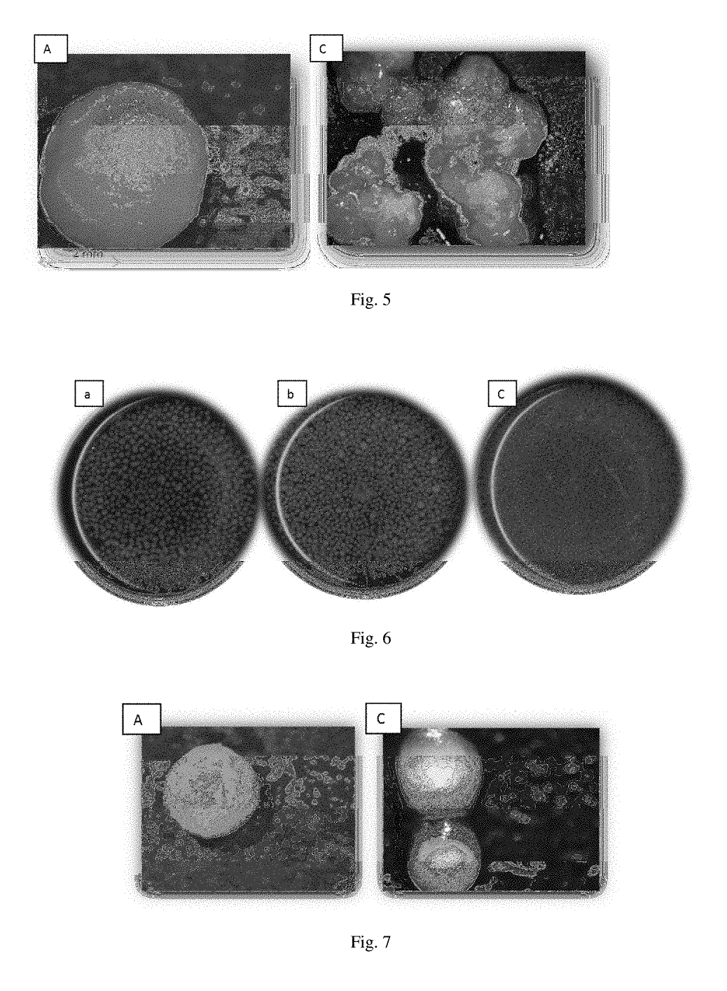

The photographs of FIG. 4 are taken by scanning the bottom of the Erlenmeyer flasks containing the fungi after 24 hours' culture. It is observed that the photographs A and B show an aggregated mycelium in the form of distinct “pellets”, in contrast to the photograph C, where the culture is fluid without any aggregation. The photographs taken with a binocular magnifier in FIG. 5 show a magnification of the culture A: magnification of a “pellet” and of the culture C: magnification of a fluid sample taken from the culture C.

The isolation of the spores during their germination by means of the method according to the invention gives rise to a large morphological change of this strain during its growth in liquid medium.

### Example 3: Application to Aspergillus terreus

The spores of Aspergillus terreus, a species which has an aggregated type morphology in the form of pellets formed by a non-coagulating mechanism during their culture in a rich liquid medium, are encapsulated according to the protocol described in Example 1.

In the same way as in Example 2, observation of the encapsulation stage with the microscope shows the presence of on average 1 spore/droplet and a droplet size of about 140 to 150 μm in diameter (2 nL).

Two controls whose spores are not encapsulated according to the method of the invention are also prepared:
a control A comprising 10 ml of YPDP medium diluted to half, seeded with 5×10⁵ spores/ml and placed in a Falcon tube under the same conditions as the recovery tube of the emulsion; and
a control B, identical to the control A, to which 20 l of oil+surfactant was added.

After carrying out the method described in Example 1, the 10 ml of destabilised emulsion according to the invention containing the germinated spores are transferred to an Erlenmeyer flask containing 40 ml of YPDP rich medium. In the same way, the two controls are also transferred to two Erlenmeyer flasks containing 40 ml of YPDP rich medium.

After 24 hours' growth, the strain of Aspergillus terreus tested (NRRL 1960) shows the morphologies illustrated in FIG. 6 and FIG. 7.

It can be seen that the morphology of the investigated strain of Aspergillus terreus is aggregated in the form of pellets in the two control cultures (without encapsulation, FIGS. 6A and B), but also after encapsulation in the form of pellets of slightly smaller diameter (FIG. 6C).

The photographs taken with a binocular magnifier in FIG. 7 show an enlargement of the culture A: enlargement of a “pellet” and of the culture C: enlargement of two “pellets”.

As can be seen in the present examples, the method according to the invention enables an encapsulation of coagulant type aggregated filamentous fungi (for example the strain Aspergillus oryzae), but does not give good results for non-coagulant type fungi (for example the strain Aspergillus terreus).

In fact, the species Aspergillus terreus belonging to the non-coagulant type is capable of aggregating after germination by aggregation of the mycelial filaments. The physical separation of the spores before and during the germination therefore does not prevent this species from exhibiting an aggregated morphology in liquid medium. Aspergillus oryzae belonging to the coagulant type aggregates only in the early stage of its development (before and during the germination), and separating the spores by this technique means aggregation is no longer possible for this species.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Method for modifying the morphology of coagulant type aggregated filamentous fungi, comprising:
a) the encapsulation of the spores of the said filamentous fungi in the dispersed phase of a water-in-oil emulsion,
b) the germination of the encapsulated spores in the said dispersed phase of the emulsion,
c) the recovery of the non-aggregated germinated spores,
the said emulsion being obtained by using a microfluidic device,
d) the culturing of the germinated spores in a liquid medium.

2. Method according to claim 1, characterised in that the filamentous fungus of coagulant type aggregated morphology is preferably chosen from the species Aspergillus oryzae, Aspergillus niger or Aspergillus nidulans, and more preferably from the strains belonging to the species Aspergillus oryzae.

3. Method according to claim 1, characterised in that the dispersed phase of the emulsion is an aqueous phase containing at least water, spores of fungus to be encapsulated in suspension in a liquid culture medium.

4. Method according to claim 1, characterised in that the oily phase of the emulsion comprises at least one biocompatible oil that does not interfere in the growth of microorganisms, preferably at least one fluorinated oil.

5. Method according to claim 4, characterised in that the oily phase comprises at least one surfactant, preferably a biocompatible surfactant.

6. Method according to claim 1, characterised in that the microfluidic device comprises a first microfluidic conduit fed with aqueous phase containing in suspension the spores to be encapsulated, and a second microfluidic conduit fed with oily phase, the first conduit opening into the second conduit and forming a fluidic junction with the latter.

7. Method according to claim 6, characterised in that the first and second microfluidic conduits have a cross-section that can be inscribed in a circle of diameter between 1 μm and 1 mm, preferably between 50 and 500 and in particular of the order of 125 μm.

8. Method according to claim 3, characterised in that the aqueous phase of the emulsion is in the form of droplets dispersed in the oily phase, the said droplets having a diameter of between 10 μm and 1 mm, preferably between 50 and 500 μm, and more preferably between about 140 and 150 μm.

9. Method according to claim 1, characterised in that the microfluidic encapsulation device comprises in addition:
a means for injecting into the first conduit of the said device, a suspension formed by the aqueous phase and by the spores to be encapsulated, the said injection means being for example a syringe with a syringe pump; and
a means for injecting into the second conduit of the said device, the oily phase, the said injection means being for example a syringe with a syringe pump.

10. Method according to claim 8, characterised in that the concentration of spores in the aqueous phase and the injection flow rates of the phases are adapted so as to have on average between 1 and 10 spores per droplet, preferably 1 spore per droplet.

11. Method according to claim 1, characterised in that the microfluidic encapsulation device comprises, in addition, an outlet microfluidic conduit allowing the recovery of the emulsion, having a cross-section that can be inscribed in a circle of diameter between 125 and 1,500 μm, preferably between 500 and 1,000 μm, and in particular of the order of 750 μm.

12. Method according to claim 1, characterised in that the germination step b) is carried out while stirring at ambient temperature, in particular between 20° and 30° C., preferably at 24° C., for the time necessary for the germination of the spores, preferably between 1 and 40 hours, preferably between 10 and 30 hours, and more preferably still about 19 hours.

13. Method according to claim 1, characterised in that the recovery step c) of the germinated spores is carried out by destabilising the emulsion, preferably by rinsing with an oily phase free of surfactant, until the concentration of surfactant is too low in order to ensure the stability of the dispersion of droplets of aqueous phase in the oily phase.

14. Method according to claim 1, characterised in that the step d) of culturing the germinated spores is carried out while stirring between 20° and 30° C., preferably 24° C., in a medium allowing the growth of filamentous microorganisms, preferably in a rich medium.

15. Method according to claim 1, characterised in that the culturing is used as preliminary step in the seeding of a bioreactor or any other facility enabling the propagation and growth of the encapsulated filamentous fungus.
